(19) United States
(12) Patent Application Publication
(10) Pub. No.: US 2026/0301124 A1
(43) Pub. Date: Oct. 1, 2026

(54) HIGH RESOLUTION SYNTHETIC MEDICAL IMAGING

(71) Applicant: Medtronic Vascular, Inc., Santa Rosa, CA (US)

(72) Inventors: James Delahunty, Galway (IE); Paul J. Coates, Corte Madera, CA (US)

(21) Appl. No.: 19/489,783

(22) PCT Filed: Jun. 18, 2024
(86) PCT No.: PCT/US2024/034421

§ 371 Date: Dec. 3, 2025

Related U.S. Application Data
(60) Provisional application No. 63/508,982, filed on Jun. 19, 2023.

(51) Int. Cl. G06T 5/50; G06T 5/60

(52) U.S. Cl. CPC G06T 5/50; G06T 5/60; G06T 2207/10081; G06T 2207/10121; G06T 2207/20216; G06T 2207/30048; G06T 2207/30101

(57) ABSTRACT

Example medical systems and techniques are disclosed. An example medical system includes memory configured to store a plurality of images, each of the plurality of images having a respective resolution. The plurality of images are generated during at least two separate medical procedures, the at least two separate medical procedures occurring during at least one of different times or different dates. The medical system includes processing circuitry communicatively coupled to the memory. The processing circuitry is configured to obtain the plurality of images. The processing circuitry is configured to generate a synthetic image based on averaging the plurality of images, the synthetic image having a higher resolution than at least a majority of the plurality of images. The processing circuitry is configured to output the synthetic image.

This application claims the benefit of U.S. Provisional Application No. 63/508,982, filed Jun. 19, 2023, the entire content of which is hereby incorporated by reference.

### TECHNICAL FIELD

This disclosure relates to the imaging such as imaging used during a medical procedure.

### BACKGROUND

During a medical procedure, a clinician may use an imaging system to be able to visualize internal anatomy of a patient. Such an imaging system may display anatomy, medical instruments, or the like, and may be used to diagnose a patient condition or assist in guiding a clinician in moving a medical instrument to an intended location inside the patient. Imaging systems may use sensors to capture image data which may be displayed during the medical procedure. Imaging systems include computed tomography (CT) scan systems, fluoroscopic systems (e.g., isocentric C-arm fluoroscopic systems), intravascular ultrasound (IVUS) systems, other ultrasound imaging systems, optical coherence tomography (OCT) fractional flow reserve (FFR) systems, magnetic resonance imaging (MRI) systems, positron emission tomography (PET) systems, as well as other imaging systems. A clinician may determine a treatment strategy based on images captured by such imaging systems.

### SUMMARY

Imagers have limited resolutions. For example, CT imagers are generally limited to a 0.3-0.4 mm resolution. In practice, fluoroscopic imagers are also typically limited to a similar resolution, so as to minimize the exposure of x-rays for the patient and clinicians attending to the patient. This resolution of 0.3-0.4 mm has a limited accuracy that may not be optimal for applications such as CT-fractional flow reserve (FFR), FFR-angiography, or for guidance on clinician decisions such as a suitable rotational atherectomy, burr sizes, balloons, stents, etc.

There may be benefits to taking a plurality of images, such as CT images, over time (weeks, months, years), as this may better enable a clinician to monitor disease progression and to allow a more complete patient assessment and development of appropriate treatment strategies, and/or changes to treatment strategies. However, CT imaging requires a contrast agent be administered to the patient and exposes the patient (and attending clinicians) to radiation. Additionally, patient radiation exposure may be considered cumulative over a lifetime, so merely requiring a minimum time period between CT imaging sessions may not address radiation exposure concerns.

In general, this disclosure is directed to various techniques and medical systems for improving a resolution of image data from medical imagers while minimizing additional radiation exposure. Example techniques include generating a synthetic or composite higher-resolution image from an average of a plurality of lower-resolution images. Such plurality of lower-resolution images may be taken over a plurality of separate medical procedures over time. Separate medical procedures should be understood to be medical procedures scheduled, from a patient point of view, to be undertaken at different times, and/or on different dates. A medical procedure scheduled, from a patient point of view, to begin at one time on one date at one facility is not to be considered separate medical procedures, even though such a medical procedure may include different steps, different clinicians, different equipment, different locations within a facility, etc.

In one example, the disclosure describes a medical system comprising: memory configured to store a plurality of images of a patient, each of the plurality of images having a respective resolution, wherein the plurality of images are generated during at least two separate medical procedures, the at least two separate medical procedures occurring during at least one of different times or different dates; and processing circuitry communicatively coupled to the memory, the processing circuitry being configured to: obtain the plurality of images; generate a synthetic image based on averaging the plurality of images, the synthetic image having a higher resolution than at least a majority of the plurality of images; and output the synthetic image.

In another example, the disclosure describes a method comprising: obtaining, by processing circuitry of a medical system, a plurality of images, each of the plurality of images having a respective resolution, wherein the plurality of images are generated during at least two separate medical procedures, the at least two separate medical procedures occurring during at least one of different times or different dates; generating, by the processing circuitry, a synthetic image based on averaging the plurality of images, the synthetic image having a higher resolution than at least a majority of the plurality of images; and outputting, by the processing circuitry, the synthetic image.

In yet another example, the disclosure describes a non-transitory computer-readable medium comprising instructions, which when executed, cause processing circuitry to obtain a plurality of images, each of the plurality of images having a respective resolution, wherein the plurality of images are generated during at least two separate medical procedures, the at least two separate medical procedures occurring during at least one of different times or different dates; generate a synthetic image based on averaging the plurality of images, the synthetic image having a higher resolution than at least a majority of the plurality of images; and output the synthetic image.

These and other aspects of the present disclosure will be apparent from the detailed description below. In no event, however, should the above summaries be construed as limitations on the claimed subject matter, which subject matter is defined solely by the attached claims.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a schematic perspective view of one example of a system for generating a synthetic high resolution image according to one or more aspects of this disclosure.

FIG. 2 is a schematic view of one example of a computing system of the system of FIG. 1.

FIG. 3 is a conceptual diagram illustrating example averaging techniques according to one or more aspects of this disclosure.

FIG. 4 is a conceptual diagram illustrating example aligning and averaging techniques according to one or more aspects of this disclosure.

FIG. 5 is a conceptual diagram illustrating example contents of a Digital Imaging and Communications in Medicine (DICOM) file for a synthetic image according to one or more aspects of this disclosure.

FIG. 6 is a flow diagram of example techniques for generating a synthetic image according to one or more aspects of this disclosure.

FIG. 7 is a conceptual diagram illustrating an example machine learning model according to one or more aspects of this disclosure.

FIG. 8 is a conceptual diagram illustrating an example training process for a machine learning model according to one or more aspects of this disclosure.

### DETAILED DESCRIPTION

As discussed above, some imagers, such as CT images, are generally limited to a lower resolution (0.3-0.4 mm) than may be desirable, particularly when some medical instrument sizes, or differences between some medical instrument sizes, are smaller than the imager resolution. Other imagers, such as fluoroscopic imagers, are generally practically limited to such a lower resolution due to concerns regarding exposure of x-rays to the patient and clinicians attending to the patient. As such, it may be desirable to improve the resolution of images from lower resolution images by generating a synthetic higher resolution image from a plurality of the lower resolution images. By generating this synthetic higher resolution image, a clinician may better be able to visualize anatomy of the patient, medical instruments, implanted medical devices, and the like, which may improve the clinician's ability to correctly diagnose a patient medical condition, to determine an appropriate medical treatment, and/or to guide the clinician during such a medical treatment.

This disclosure relates to imagers which may capture image data or images of patient anatomy. There are many types of imagers to which the techniques of this disclosure may be applicable. A fluoroscopy imager may be an imager that captures real-time video data of the movements inside a part of a body of a patient by passing X-rays through the body over a period of time. A CT imager may be an imager that combines a series of X-ray images taken from different angles around the body of the patient and uses computer processing to create cross-sectional images (slices) of the bones, blood vessels, and soft tissues inside the body. An angiography imager may use X-ray imaging to visualize blood vessels and typically involves injecting a contrast material to illuminate vessels, which may be somewhat toxic to the human body. An FFRangio procedure may involve acquiring a Fractional Flow Reserve (FFR) measurement non-invasively from an angiography imager. FFR may be a measurement to determine the ratio between the maximum achievable blood flow in a diseased coronary artery and the theoretical maximum flow in a normal coronary artery. FFR may allow a clinician to quantify severity of a coronary stenosis. The techniques of this disclosure may be applicable to these imagers and imaging techniques, as well as other imagers and imaging techniques.

Many imaging techniques require a contrast agent be administered to the patient and many imaging techniques expose the patient (and attending clinicians) to radiation. Patient radiation exposure may be considered cumulative over a lifetime, so merely requiring a minimum time period between CT imaging sessions may not address radiation exposure concerns. There may be a benefit to taking a plurality of images, such as CT images, over time (weeks, months, years), as this may better enable a clinician to monitor disease progression and to allow a more complete patient assessment and development of appropriate treatment strategies and changes to treatment strategies. However, due to the radiation exposure, it may be desirable to reduce a radiation exposure of the patient at each medical procedure during which imaging is performed. Radiation exposure may be reduced by shortening a time length of exposure of X-rays to the patient, which may result in either or both of less anatomy being covered by the captured imaging data or lower resolution images being captured. For the purposes of this disclosure, a medical procedure may include any procedure during which medical imaging occurs.

More detailed information is available in higher-resolution images than in lower-resolution images. However, similar information contained in a higher-resolution image can be reconstructed from a plurality of lower-resolution images, each of which exposes the patient to substantially less radiation than a higher-resolution scan. The techniques of this disclosure may include taking a plurality of lower resolution scans and using data analytics to generate a synthetic higher-resolution image. In some examples, the plurality of lower resolution scans may be taken over time (e.g., during a plurality of separate medical procedures), which may, not only support the generation of the synthetic higher-resolution images, but also facilitate a clinician identifying and/or tracking disease progression over time.

The techniques of this disclosure may provide improved CT image resolution, fluoroscopy image resolution, and/or other image resolution. The techniques of this disclosure may provide for more accurate CT-FFR, FFR-angiography output, and/or plaque analysis than with traditional techniques. For example, the techniques of this disclosure may aid a clinician to control expansion of a balloon or stent to a high degree of accuracy. Additionally, the techniques of this disclosure may aid a clinician to select an accurate device size for use in a medical procedure where steps between device sizes are smaller than traditional CT image resolution. For example, the difference between some balloon, stent, and/or burr sizes may be approximately 0.25 mm, which is smaller than traditional CT image resolution. For example, the techniques of this disclosure may improve the effective resolution of a CT image from 0.3 mm to 0.15 mm, which may be smaller than the burr sizes. Additionally, through the use of the techniques of this disclosure, patients may be exposed to less radiation and contrast while a clinician may still obtain an image history of the evolution of heart disease of the patient.

The techniques of this disclosure make use of the likelihood that there would be some relative movement between different image frames, both taken during one medical procedure (e.g., due to heartbeat, breathing, movement of the patient, etc.) and taken over the course of a plurality of separate medical procedures. By averaging a larger plurality of images, the techniques of this disclosure may even further improve the CT image resolution.

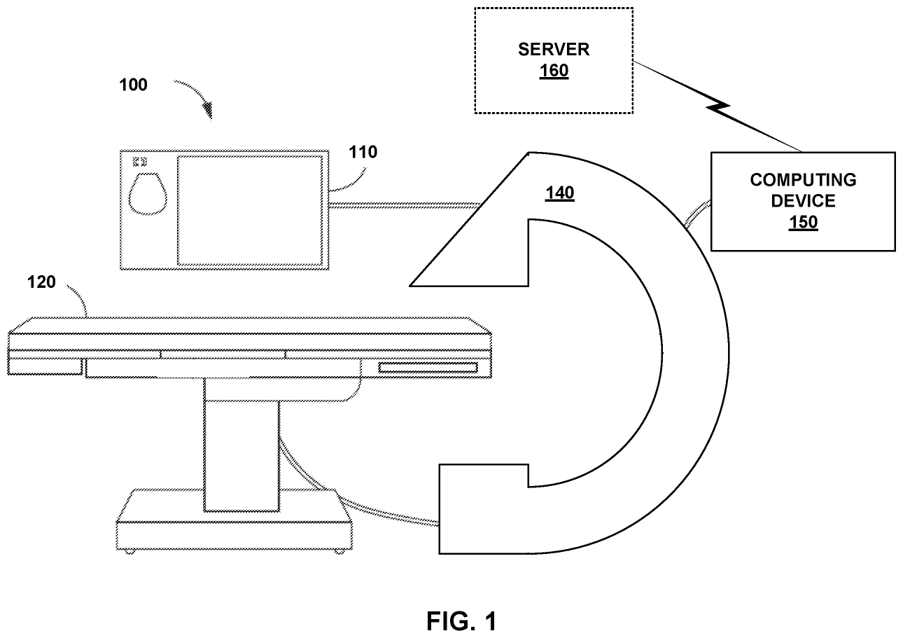

FIG. 1 is a schematic perspective view of one example of a system for generating a synthetic high resolution image according to one or more aspects of this disclosure. System 100 includes a display device 110, a table 120, an imager 140, and a computing device 150. System 100 may be an example of a system for use in an emergency room or a Cath lab. In some examples, system 100 may include other devices, not shown for simplicity purposes. In some examples, system 100 may also include server 160, which may be co-located with the other devices of system 100 or may be located elsewhere. System 100 may be used during a medical procedure, such as a diagnostic medical procedure and/or an interventional medical procedure. During such a medical procedure and over the course of a plurality of separate medical procedures, system 100 may obtain a plurality of images of anatomy of the patient, via imager 140. At least some of the images of the anatomy of the patient may be at a relatively low resolution. System 100 may generate a synthetic image based on averaging the plurality of low resolution images. This synthetic image may have a higher resolution than the plurality of low resolution images.

System 100 may include one or more machine learning models. A machine learning model may be trained to determine fiducial markers, identify fiducial markers, and/or align the low resolution images such that system 100 may average the plurality of low resolution images to generate the synthetic, higher resolution image. For example, if images are averaged without being appropriately aligned, the resulting image may be errored. For example, a machine learning model may be used to determine what to use as a fiducial marker in a plurality of images, identify that fiducial marker in at least some of the plurality of images, and/or align the at least some of the plurality of images based on the fiducial marker. After the images are appropriately aligned, system 100 may generate the synthetic image. In some examples, a machine learning model may be trained to quantify any variation in diameter of anatomy between systolic and diastolic phases in images 214 and/or in synthetic image 216, and/or in a plurality of synthetic images, such as synthetic image 216. Quantification of variation in diameters between systolic and diastolic phases of images may be useful for a clinician in monitoring disease progression.

Computing device 150 may include, for example, an off-the-shelf device such as a laptop computer, desktop computer, tablet computer, smart phone, or other similar device or may include a specific purpose device. Computing device 150 may perform various control functions with respect to imager 140. In some examples, computing device 150 may include a guidance workstation. Computing device 150 may control the operation of imager 140 and receive the output of imager 140. Computing device 150 may execute the machine learning algorithm and generate the synthetic image.

Display device 110 may be configured to output instructions, images, and messages relating to the medical procedure(s). For example, display device 110 may display any of a plurality of images obtained through imager 140 and/or the synthetic image. Table 120 may be, for example, an operating table or other table suitable for use during a medical procedure.

In the example of FIG. 1, imager 140, such as a CT imager, a fluoroscopy imager, an angiography imager, or other imaging device, may be used to image relevant portions of the patient's anatomy during a medical procedure to visualize the anatomy, characteristics and locations of lesions or other issues inside the patient's body through the generation of imaging data. While described herein primarily as a CT imager or a fluoroscopy imager, imager 140 may be any type of imaging device, such as a fluoroscopy device, a CT device, an angiography device, an intravascular ultrasound IVUS device, an OCT-FFR device, an MRI device, a PET device, an ultrasound device, or the like. In some examples, imager 140 may represent more than one imaging device, such as a plurality of any of the aforementioned devices.

Imager 140 may image a region of interest in the patient's body. The particular region of interest may be dependent on anatomy, the medical procedure, patient symptoms, and/or the like. For example, when performing a cardiac medical procedure, a portion of the vasculature and/or the heart may be within the region of interest.

Computing device 150 may be communicatively coupled to imager 140, display device 110 and/or server 160, for example, by wired, optical, or wireless communications. Server 160 may be a hospital server which may or may not be located in an emergency room or Cath lab of a hospital, a cloud-based server, or the like. Server 160 may be configured to store patient imaging data, electronic healthcare or medical records or the like. In some examples, server 160 may be configured to generate the synthetic image.

Any of, or any combination of, computing device 150, imager 140, and/or server 160 may include one or more machine learning model(s). For example, computing device 150, imager 140, and/or server 160 may obtain a plurality of images. Computing device 150, imager 140, and/or server 160 may execute one or more machine learning models to determine a fiducial marker in an image, identify the fiducial marker in other images, and/or align the images based on the fiducial marker. Once the images are aligned, computing device 150, imager 140, and/or server 160 may generate the synthetic image, for example, by averaging information within the aligned images. This synthetic image may have a higher resolution than some or all of the images used to generate the synthetic image. In some examples, computing device 150, imager 140, and/or server 160 may execute one or more machine learning models to quantify any variation in diameter between systolic and diastolic phases in images 214 and/or in synthetic image 216, and/or in a plurality of synthetic images, such as synthetic image 216.

By generating a synthetic image having a higher resolution than other images, the techniques of this disclosure may affect a particular treatment or prophylaxis for a disease or medical condition, as the techniques may reveal information otherwise unavailable within the lower resolution images, while minimizing or reducing the exposure of a patient to radiation and contrast. These techniques may improve patient outcomes, by revealing additional information, not present in a low resolution image, via the synthetic image, which may be utilized to diagnose and/or treat a medical condition of the patient.

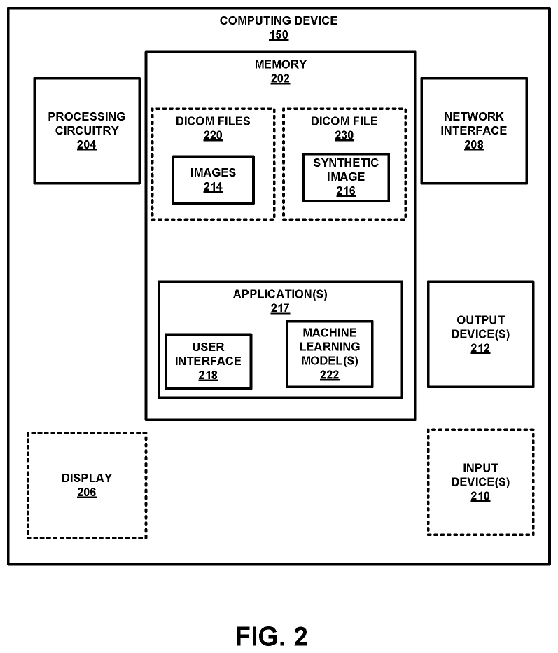

FIG. 2 is a schematic view of one example of a computing device 150 of system 10 of FIG. 1. Computing device 150 may include a workstation, a desktop computer, a laptop computer, a smart phone, a tablet, a dedicated computing device, or any other computing device capable of performing the techniques of this disclosure.

Computing device 150 may be configured to perform processing, control and other functions associated with imager 140. As shown in FIG. 2, computing device 150 may represent multiple instances of computing devices, each of which may be associated with imager 140. Computing device 150 may include, for example, a memory 202, processing circuitry 204, a display 206, a network interface 208, input device(s) 210, and/or output device(s) 212, each of which may represent any of multiple instances of such a device within the computing system, for ease of description.

While processing circuitry 204 appears in computing device 150 in FIG. 2, in some examples, features attributed to processing circuitry 204 may be performed by processing circuitry of any of computing device 150, imager 140, or server 160, or combinations thereof. In some examples, one or more processors associated with processing circuitry 204 in computing system may be distributed and shared across any combination of computing device 150, imager 140, and server 160. Computing device 150 may be used to perform any of the techniques described in this disclosure, and may form all or part of devices or systems configured to perform such techniques, alone or in conjunction with other components, such as components of computing device 150, imager 140, server 160, or a system including any or all of such systems.

Memory 202 of computing device 150 includes any non-transitory computer-readable storage media for storing data or software that is executable by processing circuitry 204 and that controls the operation of computing device 150 and/or imager 140, as applicable. In one or more examples, memory 202 may include one or more solid-state storage devices such as flash memory chips. In one or more examples, memory 202 may include one or more mass storage devices connected to the processing circuitry 204 through a mass storage controller (not shown) and a communications bus (not shown).

Although the description of computer-readable media herein refers to a solid-state storage, it should be appreciated by those skilled in the art that computer-readable storage media may be any available media that may be accessed by the processing circuitry 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium that may be used to store the desired information and that may be accessed by computing device 150. In one or more examples, computer-readable storage media may be stored in the cloud or remote storage and accessed using any suitable technique or techniques through at least one of a wired or wireless connection.

Memory 202 may store Digital Imaging and Communications in Medicine (DICOM) files 220 and DICOM file 230. DICOM files 220 may include a plurality of DICOM files each including one or more associated images 214. In some examples, images 214 may not be part of DICOM files 220, but may be stored in memory 202. DICOM file 230 may include synthetic image 216. In some examples, synthetic image 216 may not be a part of DICOM file 230, but may be stored in memory 202.

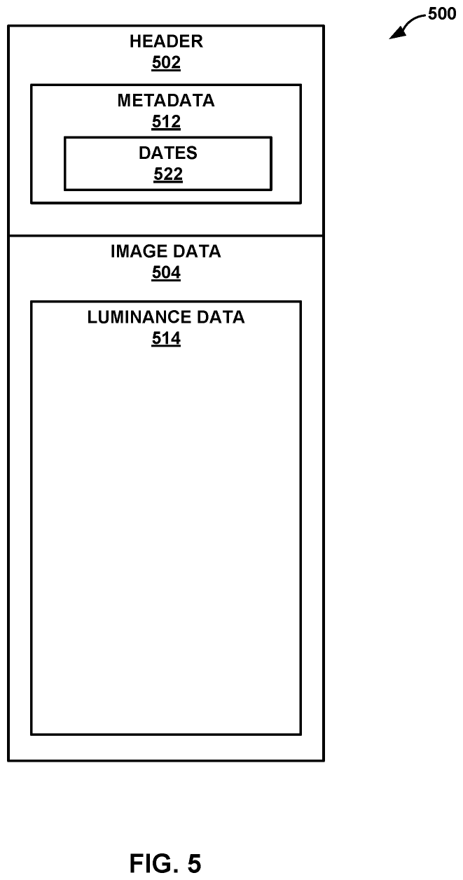

A DICOM file typically includes a header and image data. The header typically includes information about the patient and information about the image data, such as date of acquisition of the image data, equipment used to acquire the image data, size of the image data, and the like. In some examples, processing circuitry 204 may combine or average metadata from those DICOM files 220 whose images are used to generate synthetic image 216 and store such metadata in DICOM file 230. DICOM file 230 is further discussed later in this disclosure with respect to FIG. 5.

Images 214 may include a plurality of images obtained, for example, from imager 140. Images 214 may include at least some images of a relatively low (e.g., at least 0.3 mm) resolution. Synthetic image 216 may be an image generated by processing circuitry 204 based on images 214. For example, processing circuitry 204 may average images 214 to generate a higher resolution synthetic image 216.

Images 214 may be generated by imager 140 of anatomy of the patient and obtained by computing device 150 via network interface 208 which may be communicatively coupled to imager 140. In some examples, images 214 may include images generated at different times, such as during separate medical procedures. In some examples, images 214 may include images generated by imagers, such as imager 140, of different modalities (e.g., CT imager, fluoroscopic imager, etc.) and/or at different medical facilities.

For example, images 214 may be captured by imager 140 (FIG. 1) during a plurality of separate medical procedures of a patient. Processing circuitry 204 may obtain images 214 from imager 140 and store images 214 in memory 202. Processing circuitry 204 may execute user interface 218 so as to cause display 206 (and/or display device 110 of FIG. 1) to present user interface 218 to one or more clinicians performing the medical procedure.

Memory 202 may also store one or more machine learning model(s) 222 and user interface 218. Machine learning model(s) 222 may be configured to, when executed by processing circuitry 204, determine a fiducial marker in a first image of images 214, identify the fiducial marker in a remaining set of images 214, and/or align each of images 214 of the remaining set of the plurality of images and the first image based on the fiducial marker. Appropriately aligning images 214 (or at least those of images 214 including the fiducial marker) may facilitate processing circuitry 204 generating the higher resolution synthetic image 216 based on images 214. Additionally, or alternatively, machine learning model(s) 222 may be configured to, when executed by processing circuitry 204, to quantify any variation in diameter between systolic and diastolic phases in images 214 and/or in synthetic image 216, and/or in a plurality of synthetic images, such as synthetic image 216.

For example, processing circuitry 204 may use fiducial marker(s) when generating synthetic image 216. Fiducial markers may be anatomical (e.g., based on heart or vessel structure) or non-anatomical (e.g., a medical implant or the like). For example, for patients that have previously been stented, the stent itself may be used as a fiducial marker or a basis of a coordinate system. For example, processing circuitry 204 may use a wall of a vessel, a particular vessel, a valve, a sternal tie, a pacemaker, or the like, as a fiducial marker with which to align images of images 214.

In some examples, processing circuitry 204 may determine particular individual image frames of images 214 to be used to generate synthetic image 216. For example, processing circuitry 204 may generate synthetic image 216 based on based on a plurality of low resolution images of a same phase of a cardiac cycle.

Processing circuitry 204 may average information, such as pixel information, of images 214 when generating synthetic image 216. For example, processing circuitry may determine a median pixel value associated with an area of the anatomy of the patient to determine a pixel value of synthetic image 216 for that same area of the anatomy of the patient. For example, processing circuitry 204 may create a synthetic or composite higher-resolution image (e.g., synthetic image 216) from an average of a plurality of lower-resolution images (e.g., of images 214). In some examples, the plurality of lower-resolution images may be taken during a single medical procedure. In some examples, the plurality of lower-resolution images may be taken during a plurality of separate medical procedures over time. By taking images, such as images 214, over time, a clinician may track disease progression over time.

In some examples, the plurality of lower-resolution images may be part of DICOM files 220. In such a case, processing circuitry 204 may utilize the image data within the DICOM files to generate synthetic image 216.

In some examples, images 214 may include one or more relatively high resolution image. For example, imager 140 may capture one or more high resolution images of an area of particular concern or over a full area of concern (e.g., of the left main coronary or of the entire the heart). In some examples, over time, imager 140 may capture a plurality of high resolution images over different portions of an area of concern and processing circuitry 204 may generate synthetic image 216 using the plurality of high resolution images to generate a composite super-high-resolution image or may generate synthetic image 216 using the plurality of high resolution images and a plurality of low resolution images.

For example, imager 140 may capture a high-resolution image during an initial patient visit (which may be considered a medical procedure) and take only low-resolution images during subsequent visits. In such examples, processing circuitry 204 may generate synthetic image 216 based only on the low-resolution images of images 214. In such a case, a clinician may compare synthetic image 216 to the initial high-resolution image to determine disease progression.

In some examples, the low-resolution images include imaging data of a portion of the heart that is different than the represented in the initial high-resolution image and/or a portion of the heart that overlaps with imaging data of the initial high-resolution image. In some examples, each of the low-resolution images includes imaging data of a different portion of the heart. In some examples, the section of the heart to be included in a given low resolution image is determined prior to a respective medical procedure and is based on external data (e.g., electrocardiogram (ECG) data indicative of a potential specific issue, for example, from a stress test.). In some examples, the section of the heart to be included in a given low resolution image is the Left Main (LM), as undiagnosed LM disease has greater likelihood of being fatal.

Processing circuitry 204 may be implemented by one or more processors, which may include any number of fixed-function circuits, programmable circuits, or a combination thereof. In various examples, control of any function by processing circuitry 204 may be implemented directly or in conjunction with any suitable electronic circuitry appropriate for the specified function. Fixed-function circuits refer to circuits that provide particular functionality and are preset on the operations that may be performed. Programmable circuits refer to circuits that may programmed to perform various tasks and provide flexible functionality in the operations that may be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Instructions may be executed by one or more processors, such as one or more digital signal processors (DSPs), general purpose microprocessors, application specific integrated circuits (ASICs), field programmable gate arrays (FPGAs), graphics processing units (GPUs) or other equivalent integrated or discrete logic circuitry. Accordingly, the term processing circuitry 204 as used herein may refer to one or more processors having any of the foregoing processor or processing structure or any other structure suitable for implementation of the techniques described herein. In addition, in some aspects, the functionality described herein may be provided within dedicated hardware or software modules configured for encoding and decoding, or incorporated in a combined codec. Also, the techniques could be fully implemented in one or more circuits or logic elements.

Display 206 may be touch sensitive or voice activated, enabling display 206 to serve as both an input and output device. Alternatively, a keyboard (not shown), mouse (not shown), or other data input devices (e.g., input device(s) 210) may be employed.

Network interface 208 may be adapted to connect to a network such as a local area network (LAN) that includes a wired network or a wireless network, a wide area network (WAN), a wireless mobile network, a Bluetooth network, or the internet. For example, computing device 150 may obtain images 214 from imager 140 during a medical procedure. Computing device 150 may receive updates to its software, for example, application(s) 217, via network interface 208. Computing device 150 may also display notifications on display 206 that a software update is available.

Input device(s) 210 may include any device that enables a user to interact with computing device 150, such as, for example, a mouse, keyboard, foot pedal, touch screen, augmented-reality input device receiving inputs such as hand gestures or body movements, or voice interface.

Output device(s) 212 may include any connectivity port or bus, such as, for example, parallel ports, serial ports, universal serial busses (USB), or any other similar connectivity port known to those skilled in the art.

Application(s) 217 may be one or more software programs stored in memory 202 and executed by processing circuitry 204 of computing device 150. Processing circuitry 204 may execute user interface 218, which may display images 214, synthetic image 216, DICOM files 220, and/or DICOM file 230 on display 206 and/or display device 110. A clinician may use the displayed images or files to make a diagnosis, track a progress of a medical condition over time, determine a treatment strategy, monitor the progress of an interventional medical procedure, or the like.

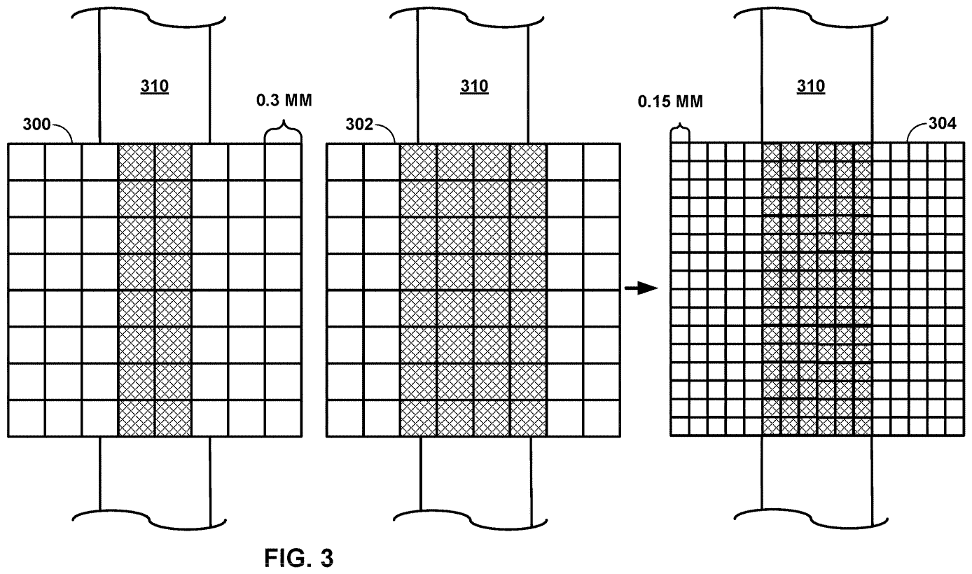

FIG. 3 is a conceptual diagram illustrating example averaging techniques according to one or more aspects of this disclosure. Image 300 represents a first image captured of a portion of vessel 310. Image 302 represents a second image captured of a same portion of vessel 310 as image 300. Images 300 and 302 may be examples of two images of images 214 (FIG. 2). Each square within images 300 and 302 represents a pixel with a resolution of 0.3 mm. Images 300 and 302 depicts those pixels indicative of a vessel wall in using cross hatching. For the purposes of FIG. 3, images 300 and 302 are generally aligned, for example, based on a fiducial marker which may be used to align images 300 and 302 (not shown in FIG. 3). As can be seen, in image 300 the borders of vessel 310 are detected in a different location than the borders of vessel 310 in image 302.

Image 304 may represent a synthetic image (e.g., an example of synthetic image 216 of FIG. 2) based on images 300 and 302. Image 304 may have a higher resolution than image 300 and image 302, at 0.15 mm per pixel. As such the vessel wall of vessel 310 may be more accurately located as shown in image 304.

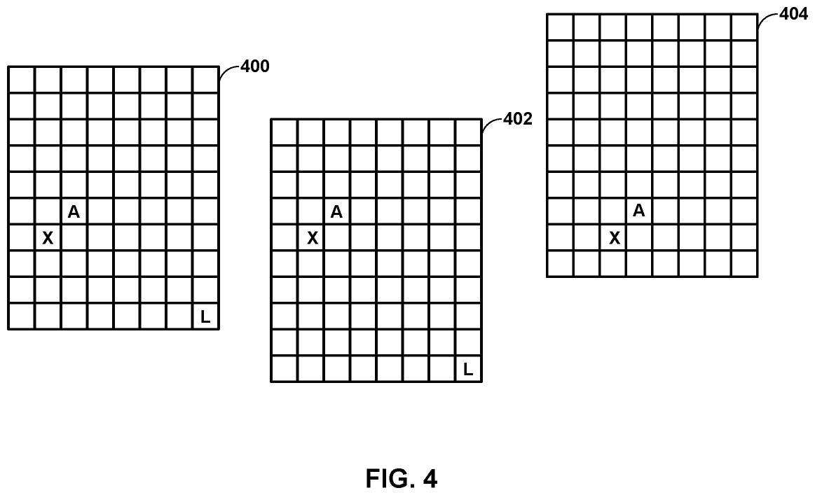

FIG. 4 is a conceptual diagram illustrating example aligning and averaging techniques according to one or more aspects of this disclosure. Images 400, 402, and 404 may be example images of plurality of images 214 (FIG. 2). Each of images 400, 402, and 404 are depicted having a same resolution, with each small square within each image representing a pixel. Image 402 is depicted as vertically offset from image 400. Image 404 is depicted as being both vertically and horizontally offset from image 400. It should be noted that any of plurality of images 214 may be offset, vertically, horizontally, or both vertically and horizontally, from each other. In some examples, one or more of plurality of images 214 may be substantially offset from each other such that a fiducial marker represented in one image may not be represented in another image.

Processing circuitry 204 may determine a fiducial marker (marked with an X) in image 400. While shown as an individual pixel for purposes of illustration, it should be understood that a fiducial marker may be larger than a single pixel, for example, a fiducial marker may occupy a plurality of pixels.

Processing circuitry 204 may identify the fiducial marker X in image 402. Image 402 may be offset from image 400, for example, in a vertical direction as shown. As such a lowest most right pixel L of image 400 would not represent the same anatomy as the lowest most right pixel L of image 402, which would represent anatomy not even present in image 400. Therefore, averaging the pixels L would result in a blurring or distortion of image 400 and image 402 rather than a synthesized image of higher resolution than images 400 and 402.

Processing circuitry 204 may also identify the fiducial marker X in image 404. Image 404 may be offset from image 400, for example, in a horizontal direction and vertical direction as shown. Processing circuitry 204 may identify a pixel A in relation to the fiducial marker X (e.g., one pixel above and one pixel to the right of fiducial marker X) that is represented in more than one of the plurality of images 214 (FIG. 2). For example, pixel A is represented in each of images 400, 402, and 404. Processing circuitry 204 may average the pixel as from the three images 400, 402, and 404. For example, processing circuitry 204 may determine a median value of pixel A across images 400, 402, and 404, which processing circuitry 204 may use as a value of an associated pixel A in a synthetic image, such as synthetic image 216. Processing circuitry 204 may perform the same techniques for each pixel relative to fiducial marker X. In some instances, a pixel may appear in less than all of images 400, 402, and 404 due to the offset of the images from each other. In such cases, processing circuitry 204 may perform the averaging for such a pixel without including the image(s) in which that particular pixel does not appear. For example, processing circuitry 204 may determining a median value of respective pixels across the plurality of images 400, 402, and 404 (or the plurality of images 214) to generate an average pixel value for corresponding pixels so as to generate each of the pixels of synthetic image 216.

FIG. 5 is a conceptual diagram illustrating example contents of a DICOM file for a synthetic image according to one or more aspects of this disclosure. DICOM file 500 may be an example of DICOM file 230 (FIG. 2). DICOM file 500 may include a header 502 and image data 504. Header 502 may include metadata 512. Metadata 512 may include information associated with image data 504, such as patient identification information, information related to modality, settings, date associated with image data 504. Metadata 512 may include dates 522, which may include date information of when each image processing circuitry 204 used to generate synthetic image 216 was generated. For example, if processing circuitry 204 used images of plurality of images 214 generated by an imager, such as imager 140, on Jan. 31, 2020, and Jul. 1, 2021, dates 522 would include both Jan. 31, 2020, and Jul. 1, 2021. As such, metadata 512 may preserve and include information, such as date, associated with each image used to generate synthetic image 216. Dates 522 may also include a date on which synthetic image 216 was generated.

Image data 504 may include synthetic image 216. In some examples, image data 504 may be stored in a standardized format, such as JPEG, TIFF, GIF, PNG, etc. Image data 504 may include luminance data 514. For example, each pixel of synthetic image 216 may have an associated luminance which may be stored in luminance data 514.

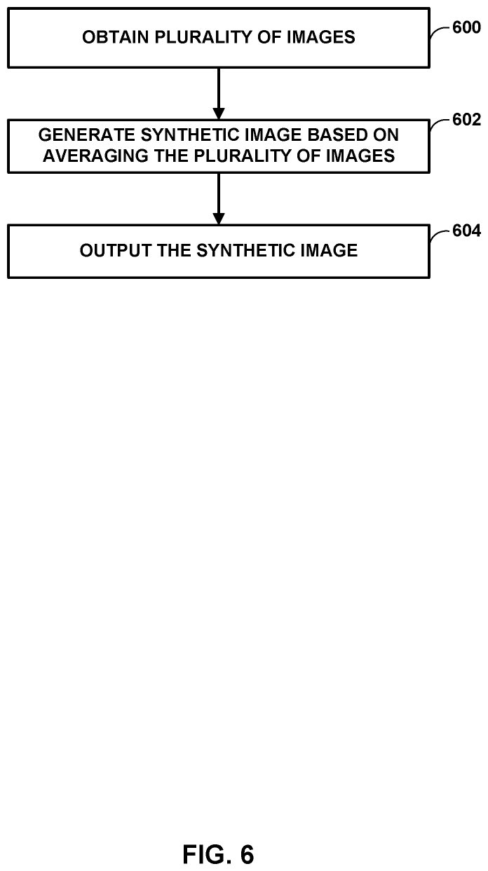

FIG. 6 is a flow diagram of example techniques for generating a synthetic image according to one or more aspects of this disclosure. The techniques of FIG. 6 are described below with respect to processing circuitry 204, but such techniques may be performed by any of, or any combination of, processing circuitry of devices depicted in FIG. 1 or capable of performing such techniques.

Processing circuitry 204 may obtain the plurality of images (600). For example, processing circuitry 204 may retrieve plurality of images 214 from memory 202 and/or receive plurality of images 214 from imager 140 or server 160 via network interface 208. Each of the plurality of images has a respective resolution and the plurality of images are generated during at least two separate medical procedures, the at least two separate medical procedures occurring during at least one of different times or different dates. For example, the separate medical procedures may be separated by time, such as hours, days, weeks, months, years, or the like. It should be understood that different steps of a medical procedure scheduled for the patient to arrive for the procedure at a specific time on a specific date are not to be considered separate medical procedures, even if the different steps include different clinicians, equipment, locations, or the like.

Processing circuitry 204 may generate a synthetic image based on averaging the plurality of images, the synthetic image having a higher resolution than at least a majority of the plurality of images (602). A majority may be greater than 50 percent. For example, processing circuitry 204 may generate synthetic image 216 by averaging (e.g., determining a median) pixel values of pixels of plurality of images 214. In this manner, processing circuitry 204 may generate synthetic image 216 which may be of a relatively higher resolution than plurality of images 214.

Processing circuitry 204 may output the synthetic image (604). For example, processing circuitry 204 may output synthetic image 216 to display 206 and/or display device 110 for display or output synthetic image 216 to server 160 (FIG. 1) for storage. A clinician may review the synthetic image 216 for diagnostic and/or therapeutic purposes.

In some examples, processing circuitry 204 is configured to determine a fiducial marker (e.g., fiducial marker X) in a first image (e.g., image 400) of the plurality of images 214. In such examples, processing circuitry 204 may identify the fiducial marker in a remaining set of the plurality of images (e.g., images 402 and 404). In such examples, processing circuitry 204 may align each of the images of the remaining set of the plurality of images and the first image based on the fiducial marker. In some examples, processing circuitry 204 is configured to execute machine learning model(s) 222 to at least one of determine the fiducial marker, identify the fiducial marker, or align each of the images of the remaining set of the plurality of images and the first image. In some examples, machine learning model(s) 222 is trained on images of anatomy of the current patient, images of anatomy of other patients, and/or the like. Training data may include image data from past medical procedures performed on a plurality of patients having different patient conditions, different prior medical procedures, annotations or tags associated with image data, or the like.

In some examples, aligning each of the images of the remaining set of the plurality of images and the first image causes respective pixels (e.g., pixel A) of each of the images of the plurality of images to be aligned, and wherein averaging the plurality of images comprises determining a median value of the respective pixels across the plurality of images. In some examples, the respective pixels comprise luminance information (e.g., luminance data 514).

In some examples, processing circuitry 204 is configured to execute machine learning model(s) 222 to determine a variation in diameter of anatomy between systolic and diastolic phases of anatomy of the patient based on at least one of images 214 or synthetic image 216.

In some examples, at least one of the plurality of images has a higher resolution than a remainder of the plurality of images. In some examples, the at least one of the plurality of images was generated during a first in time of the at least two separate medical procedures. In some examples, the at least one of the plurality of images comprises image data of a region of interest of the patient, the region of interest being identified as a region associated with a relatively higher risk than other regions of the patient.

In some examples, each of the plurality of images is contained within a respective Digital Imaging and Communications in Medicine (DICOM) file (e.g., DICOM files 220) stored in the memory, and wherein as part of obtaining the plurality of images, the processing circuitry is configured to extract plurality of images 214 from the respective DICOM files. In some examples, processing circuitry 204 is further configured to generate a DICOM file (e.g., DICOM file 230) comprising synthetic image 216 and at least one of store the DICOM file in the memory or output to DICOM file to a device for storage. In some examples, processing circuitry 204 is further configured to generate metadata (e.g., metadata 512) associated with the synthetic image, the metadata being based on metadata associated with the plurality of images. In some examples, the metadata associated with the synthetic image comprises a plurality of dates (e.g., dates 522) corresponding to dates of at least two separate medical procedures. In some examples, processing circuitry 204 is further configured to insert the metadata associated with the synthetic image into a Digital Imaging and Communications in Medicine (DICOM) file.

In some examples, the plurality of images are based on a plurality of imaging modalities. In some examples, at least one of the plurality of images was generated using at least one of fluoroscopy or computed tomography.

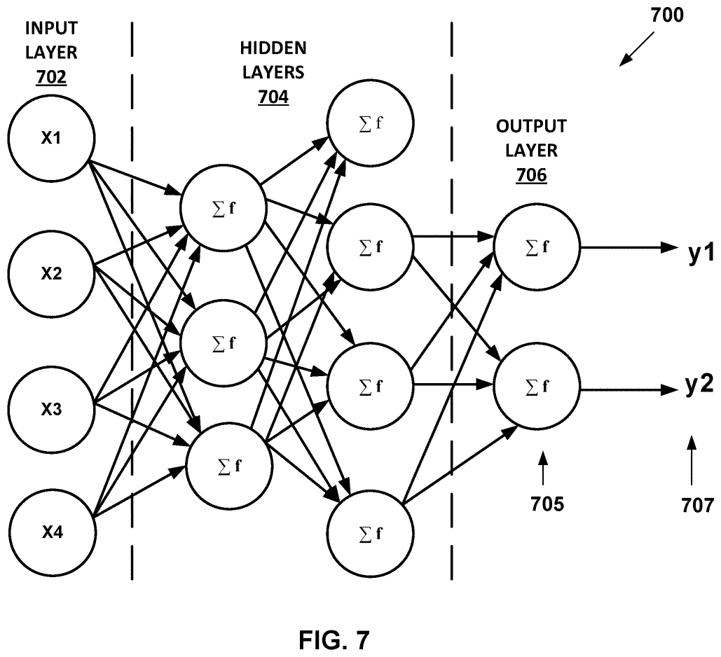

FIG. 7 is a conceptual diagram illustrating an example machine learning model according to one or more aspects of this disclosure. Machine learning model 700 may be an example of machine learning model(s) 222. Machine learning model 700 may be an example of a deep learning model, or deep learning algorithm, trained to determine an identification of an implanted medical device and/or to determine a recommended treatment strategy. One or more of computing device 150 and/or server 160 may train, store, and/or utilize machine learning model 700, but other devices of system 100 may apply inputs to machine learning model 700 in some examples. In some examples, other types of machine learning and deep learning models or algorithms may be utilized in other examples. For examples, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

As shown in the example of FIG. 7, machine learning model 700 may include three types of layers. These three types of layers include input layer 702, hidden layers 704, and output layer 706. Output layer 706 comprises the output from the transfer function 705 of output layer 706. Input layer 702 represents each of the input values X1 through X4 provided to machine learning model 700. In some examples, the input values may include any of the values input into the machine learning model, as described above. For example, the input values may include images 214, as described above. In addition, in some examples input values of machine learning model 700 may include additional data, such as other data that may be collected by or stored in system 100.

Each of the input values for each node in the input layer 702 is provided to each node of a first layer of hidden layers 704. In the example of FIG. 7, hidden layers 704 include two layers, one layer having four nodes and the other layer having three nodes, but fewer or greater number of nodes may be used in other examples. Each input from input layer 702 is multiplied by a weight and then summed at each node of hidden layers 704. During training of machine learning model 700, the weights for each input are adjusted to establish a relationship between images 214, potential fiducial markers which may be found in one or more of images 214, and/or an alignment of images 214 based on one or more fiducial markers. In some examples, one hidden layer may be incorporated into machine learning model 700, or three or more hidden layers may be incorporated into machine learning model 700, where each layer includes the same or different number of nodes.

The result of each node within hidden layers 704 is applied to the transfer function of output layer 706. The transfer function may be linear or non-linear, depending on the number of layers within machine learning model 700. Example non-linear transfer functions may be a sigmoid function or a rectifier function. The output 707 of the transfer function may be a classification that images 214 and/or 3D synthetic image 216 is indicative of a particular implanted medical device and/or a particular recommended treatment strategy.

As shown in the example above, by applying machine learning model 700 to input data such as images 214, processing circuitry 204 is able to determine a fiducial marker in one image, identify the fiducial marker in other images, and/or align the images based on the fiducial marker. This may improve the ability of processing circuitry 204 to generate synthetic image 216 as a higher resolution image than one or more of images 214. Additionally, or alternatively, by applying machine learning model 700 to input data such as images 214 and/or synthetic images, such as synthetic image 216, processing circuitry 204 is able to quantify any variation in diameter between systolic and diastolic phases of patient anatomy represented in such images.

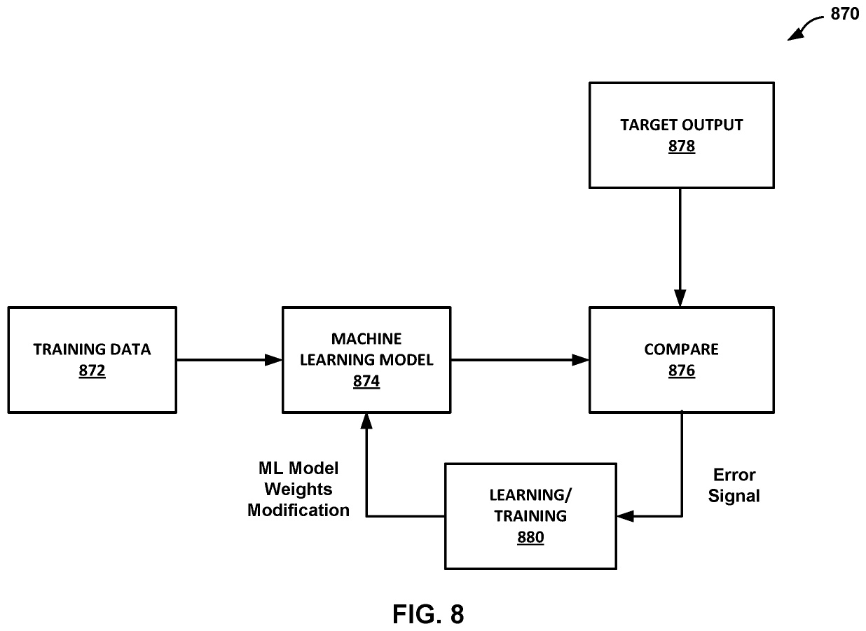

FIG. 8 is a conceptual diagram illustrating an example training process for a machine learning model according to one or more aspects of this disclosure. Process 870 may be used to train machine learning model(s) 222 or machine learning model 700. A machine learning model 874 (which may be an example of machine learning model 700 and/or machine learning model(s) 222) may be implemented using any number of models for supervised and/or reinforcement learning, such as but not limited to, an artificial neural network, a decision tree, naïve Bayes network, support vector machine, or k-nearest neighbor model, convolutional neural network (CNN), regional neural network (RNN), long short-term memory (LSTM), ensemble network, to name only a few examples.

In some examples, one or more of computing device 150 and/or server 160 initially trains machine learning model 874 based on a corpus of training data 872. Training data 872 may include, for example, images of anatomy of the current patient, images of anatomy of other patients, and/or the like. In some examples, training data 872 may include annotations identifying one or more fiducial markers and/or anatomy contained in the images of training data 872. Training data 872 may include data from past medical procedures performed on a plurality of patients having different patient conditions, different prior medical procedures, annotations or tags, other training data mentioned herein, and/or the like. In some examples, training data 872 may include images of varying resolutions, including lower resolution images and higher resolution images.

While training machine learning model 874, processing circuitry of system 100 may compare 876 a prediction or classification with a target output 878. Processing circuitry 204 may utilize an error signal from the comparison to train (learning/training 880) machine learning model 874. Processing circuitry 204 may generate machine learning model weights or other modifications which processing circuitry 204 may use to modify machine learning model 874. For examples, processing circuitry 204 may modify the weights of machine learning model 874 based on the learning/training 880. For example, one or more of computing device 150 and/or server 160, may, for each training instance in training data 872, modify, based on training data 872, the manner in which a fiducial marker is determined, identified, and/or images are aligned and/or the manner in which a diameter of anatomy between systolic and diastolic phases is quantified.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The terms “controller”, “processor”, or “processing circuitry” may generally refer to any of the foregoing logic circuitry, alone or in combination with other logic circuitry, or any other equivalent circuitry. A control unit comprising hardware may also perform one or more of the techniques of this disclosure. Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), or electronically erasable programmable read only memory (EEPROM), or other computer readable media.

This disclosure includes the following non-limiting examples.

Example 1. A medical system comprising: memory configured to store a plurality of images of a patient, each of the plurality of images having a respective resolution, wherein the plurality of images are generated during at least two separate medical procedures, the at least two separate medical procedures occurring during at least one of different times or different dates; and processing circuitry communicatively coupled to the memory, the processing circuitry being configured to: obtain the plurality of images; generate a synthetic image based on averaging the plurality of images, the synthetic image having a higher resolution than at least a majority of the plurality of images; and output the synthetic image.

Example 2. The medical system of example 1, wherein the processing circuitry is further configured to: determine a fiducial marker in a first image of the plurality of images; identify the fiducial marker in a remaining set of the plurality of images; and align each of the images of the remaining set of the plurality of images and the first image based on the fiducial marker.

Example 3. The medical system of example 2, wherein the processing circuitry is configured to execute a machine learning model to at least one of determine the fiducial marker, identify the fiducial marker, or align each of the images of the remaining set of the plurality of images and the first image.

Example 4. The medical system of example 3, wherein the machine learning model is trained on a plurality of past images.

Example 5. The medical system of any of examples 2-4, wherein the aligning each of the images of the remaining set of the plurality of images and the first image causes respective pixels of each of the images of the plurality of images to be aligned, and wherein averaging the plurality of images comprises determining a median value of the respective pixels across the plurality of images.

Example 6. The medical system of example 5, wherein the respective pixels comprise luminance information.

Example 7. The medical system of any of examples 1-6, wherein the processing circuitry is configured to execute a machine learning model to determine a variation in diameter of anatomy between systolic and diastolic phases of anatomy of the patient based on at least one of the plurality of images or the synthetic image.

Example 8. The medical system of any of examples 1-7, wherein at least one of the plurality of images has a higher resolution than a remainder of the plurality of images.

Example 9. The medical system of example 8, wherein the at least one of the plurality of images was generated during a first in time of the at least two separate medical procedures.

Example 10. The medical system of example 8 or example 9, wherein the at least one of the plurality of images comprises image data of a region of interest of the patient, the region of interest being identified as a region associated with a relatively higher risk than other regions of the patient.

Example 11. The medical system of any of examples 1-10, wherein each of the plurality of images is contained within a respective Digital Imaging and Communications in Medicine (DICOM) file stored in the memory, and wherein as part of obtaining the plurality of images, the processing circuitry is configured to extract the plurality of images from the respective DICOM files.

Example 12. The medical system of example 11, wherein the processing circuitry is further configured to: generate a DICOM file comprising the synthetic image; and at least one of store the DICOM file in the memory or output to DICOM file to a device for storage.

Example 13. The medical system of example 12, wherein the processing circuitry is further configured to generate metadata associated with the synthetic image, the metadata being based on metadata associated with the plurality of images.

Example 14. The medical system of example 13, wherein the metadata associated with the synthetic image comprises a plurality of dates corresponding to dates of the at least two separate medical procedures.

Example 15. The medical system of example 14, wherein the processing circuitry is further configured to insert the metadata associated with the synthetic image into a Digital Imaging and Communications in Medicine (DICOM) file.

Example 16. The medical system of any of examples 1-15, wherein the plurality of images are based on a plurality of imaging modalities.

Example 17. The medical system of any of examples 1-16, wherein at least one of the plurality of images was generated using at least one of fluoroscopy or computed tomography.

Example 18. The medical system of any of examples 1-17, further comprising a display, wherein the processing circuitry is configured to output the synthetic image for display on the display.

Example 19. A method comprising: obtaining, by processing circuitry of a medical system, a plurality of images, each of the plurality of images having a respective resolution, wherein the plurality of images are generated during at least two separate medical procedures, the at least two separate medical procedures occurring during at least one of different times or different dates; generating, by the processing circuitry, a synthetic image based on averaging the plurality of images, the synthetic image having a higher resolution than at least a majority of the plurality of images; and outputting, by the processing circuitry, the synthetic image.

Example 20. A non-transitory computer-readable storage medium storing instructions, which when executed cause processing circuitry to: obtain a plurality of images, each of the plurality of images having a respective resolution, wherein the plurality of images are generated during at least two separate medical procedures, the at least two separate medical procedures occurring during at least one of different times or different dates; generate a synthetic image based on averaging the plurality of images, the synthetic image having a higher resolution than at least a majority of the plurality of images; and output the synthetic image.

Various examples have been described. These and other examples are within the scope of the following claims.

1. A medical system for generating a synthetic image, the medical system comprising:
memory configured to store a plurality of images of a patient, each of the plurality of images having a respective resolution, wherein the plurality of images are generated during at least two separate medical procedures, the at least two separate medical procedures occurring during at least one of different times or different dates; and
processing circuitry communicatively coupled to the memory, the processing circuitry being configured to:
obtain the plurality of images;
generate a synthetic image based on averaging the plurality of images, the synthetic image having a higher resolution than at least a majority of the plurality of images; and
output the synthetic image.

2. The medical system of claim 1, wherein the processing circuitry is further configured to:
determine a fiducial marker in a first image of the plurality of images;
identify the fiducial marker in a remaining set of the plurality of images; and
align each of the images of the remaining set of the plurality of images and the first image based on the fiducial marker.

3. The medical system of claim 2, wherein the processing circuitry is configured to execute a machine learning model to at least one of determine the fiducial marker, identify the fiducial marker, or align each of the images of the remaining set of the plurality of images and the first image.

4. The medical system of claim 3, wherein the machine learning model is trained on a plurality of past images.

5. The medical system of claim 2, wherein the aligning each of the images of the remaining set of the plurality of images and the first image causes respective pixels of each of the images of the plurality of images to be aligned, and wherein averaging the plurality of images comprises determining a median value of the respective pixels across the plurality of images.

6. The medical system of claim 5, wherein the respective pixels comprise luminance information.

7. The medical system of claim 1, wherein the processing circuitry is configured to execute a machine learning model to determine a variation in diameter of anatomy between systolic and diastolic phases of anatomy of the patient based on at least one of the plurality of images or the synthetic image.

8. The medical system of claim 1, wherein at least one of the plurality of images has a higher resolution than a remainder of the plurality of images.

9. The medical system of claim 8, wherein the at least one of the plurality of images was generated during a first in time of the at least two separate medical procedures.

10. The medical system of claim 8, wherein the at least one of the plurality of images comprises image data of a region of interest of the patient, the region of interest being identified as a region associated with a relatively higher risk than other regions of the patient.

11. The medical system of claim 1, wherein each of the plurality of images is contained within a respective Digital Imaging and Communications in Medicine (DICOM) file stored in the memory, and wherein as part of obtaining the plurality of images, the processing circuitry is configured to extract the plurality of images from the respective DICOM files.

12. The medical system of claim 11, wherein the processing circuitry is further configured to:
generate a DICOM file comprising the synthetic image; and
at least one of store the DICOM file in the memory or output to DICOM file to a device for storage.

13. The medical system of claim 12, wherein the processing circuitry is further configured to generate metadata associated with the synthetic image, the metadata being based on metadata associated with the plurality of images.

14. A method for generating a synthetic image, the method comprising:
obtaining, by processing circuitry of a medical system, a plurality of images, each of the plurality of images having a respective resolution, wherein the plurality of images are generated during at least two separate medical procedures, the at least two separate medical procedures occurring during at least one of different times or different dates;
generating, by the processing circuitry, a synthetic image based on averaging the plurality of images, the synthetic image having a higher resolution than at least a majority of the plurality of images; and
outputting, by the processing circuitry, the synthetic image.

15. A non-transitory computer-readable storage medium storing instructions, which when executed cause processing circuitry to generate a synthetic image by causing the processing circuitry to:
obtain a plurality of images, each of the plurality of images having a respective resolution, wherein the plurality of images are generated during at least two separate medical procedures, the at least two separate medical procedures occurring during at least one of different times or different dates;
generate a synthetic image based on averaging the plurality of images, the synthetic image having a higher resolution than at least a majority of the plurality of images; and
output the synthetic image.

16. The medical system of claim 13, wherein the metadata associated with the synthetic image comprises a plurality of dates corresponding to dates of the at least two separate medical procedures.

17. The medical system of claim 16, wherein the processing circuitry is further configured to insert the metadata associated with the synthetic image into the DICOM file

18. The medical system of claim 1, wherein the plurality of images are based on a plurality of imaging modalities.

19. The medical system of claim 1, wherein at least one of the plurality of images was generated using at least one of fluoroscopy or computed tomography.

20. The medical system of claim 1, further comprising a display, wherein the processing circuitry is configured to output the synthetic image for display on the display.
